(19) United States
(12) Patent Application Publication
(10) Pub. No.: US 2020/0278311 A1
(43) Pub. Date: Sep. 3, 2020

(54) PLASTIC FILM SENSOR

(71) Applicant: SEALWACS AB, Sävedalen (SE)

(72) Inventor: Erik ISBERG, Bjästa (SE)

(73) Assignee: SEALWACS AB, Sävedalen (SE)

(21) Appl. No.: 16/649,291

(22) PCT Filed: Sep. 26, 2018
(86) PCT No.: PCT/SE2018/000022

§ 371 Date: Mar. 20, 2020

(30) Foreign Application Priority Data
Oct. 6, 2017 (SE) 1700240-3

(51) Int. Cl. G01N 27/20; G01N 33/44; B29C 65/00; B29C 65/82; B29C 65/22

(52) U.S. Cl. CPC G01N 27/205; G01N 33/442; B29C 66/8511; B29L 2031/7128; B29C 65/22; B29C 66/4312; B29C 65/8276

(57) ABSTRACT

A sensor for real time monitoring the quality of welding joints in the plastic film at the production of plastic bags on roll by measuring material changes in the welding joint is provided. The sensor comprises at least two electrode pairs arranged to measure changes in the dielectric properties of the material when the welding joint is passing through the measuring gap of the electrodes and provide a measuring signal proportional to the material change in the welding joint. The measuring signal is evaluated to detect defective welding joints in the plastic film. The electrodes are arranged so that one pair of the electrodes constitutes the signal measuring gap arranged for measuring the welding profile. The form of the measuring electrode conforms to the direction and form of the welding joint. The other pair of electrodes constitutes the reference measuring gap arranged to provide an averaged signal across the surface of the plastic film. The difference between the reference signal and the measuring signal then provides a signal which can be used for monitoring the welding quality.

The present invention relates to a sensor for monitoring in real time the quality of welding joints in the production of plastic bags on a roll. The sensor is arranged to be mounted at the side of the plastic film, along the production line to measure material changes in the welding joint. The sensor comprises at least two measuring gaps with each two measuring electrodes. In the measuring gaps changes in the properties of the dielectric material are measured as the welding joint passes through the measuring gaps of the electrodes and a sensor signal proportional to the change of material in the welding joint is provided. The sensor further comprises signal processing means for evaluating the measuring signal in order to detect poor quality welding joints of the plastic film.

Since the 1950th the manufacturing of plastic bags has developed into a highly automated industry. Such development has taken place in all manufacturing industry and wherein the focus has been to “build away” any quality problems by a continuous improvement of the manufacturing process. From an economical point of view the plastic bag industry is characterized by relatively low profit margins, normally around a few percent, which has led the industry for a long time to strive to rationalize the manufacturing process. A high degree of automation means a very rapid physical flow. Machine operators today are responsible for a number of production lines running simultaneously. The production of plastic film is affected by a number of factors, such as feed rate, melting temperature, ambient temperature in the room, quality of the material and thickness. Some factors are difficult to measure and influence. For example, the granulate might to some extent absorb water which affects the manufacturing process. The above factors also affect the quality of the welding joint in plastic bags. Furthermore, mechanical changes in the welding station might occur through wear or damage which also means that the welding joint might be impaired to the worse. Bad quality plastic bags are collected and re-granulated.

With traditional process improvements the industry has tried to reduce the margin of error, i.e. to reduce the proportion of defective products in relation to the total manufactured quantity. However, the industry has lacked a technical solution to monitor and control the quality of welding joints in real time, i.e. continuously during the manufacturing process. Instead, the control usually takes place on a sample basis by the machine operator blowing up a plastic bag and then squeezing the bag until it breaks. The experience of the individual machine operator of the welding joint quality in relation to the pressure on the bag then determines if the bag maintains the desired quality. The sample pick ups are usually made at intervals of an hour or so. When the machine operator has found quality defects the production line is stopped and search is started back along the production chain to find out where the bad quality first appeared. In bad case, the quality might have broken just a few minutes after the last check which means that the operator has to break up packages and test plastic bags for almost one hour production back in time.

When the fault that caused the quality defect has been found, it will be rectified. Other production lines might be running but without a direct supervision from the operator who have been busy with troubleshooting. Thus there is an evident risk that defects in other production lines have continued during the time the operator was busy with the first fault. Such manual and time consuming control and sorting out bad quality bags is the main reason why there is a significant disposal cost for the manufacturer. Already at low levels of rejection there is a high cost involved, specifically due to machine time lost when a defective product has been manufactured, operators time, material that needs re-granulation and destruction of packaging. The manufacturing cost of disposal might be estimated to approximately ten percent of the total production cost, although the physical disposal is only some single percentage. In the absence of a more efficient solution, this is a condition that the industry has been forced to accept so far. For a low margin product, it is obvious that the level of disposal has a very big impact on the profitability of the product. As the operator has to go back manually into the production chain and search for products with inadequate quality, it is a clear risk that not all defects are identified. As a result, the producer risks that, in addition to the financial aspect, bad will also occurs in cases where poor products reach their customers, which in the long run can lead to customer loss. When bags produced with new raw materials have to be scrapped due to a defective welding joint, they are re-cycled by re-granulation. Then the material value is decreased by up to 50% and is usually used only to produce low-price bags, such as garbage bags.

The problem of bad welding joints has thus been known in the industry for a long time, but a satisfactory solution to this problem has been missing. However, many different solutions have been implemented. One reason why no satisfactory solution has been identified so far might be the fact that modern industry has more focused on process improvements instead of control and monitoring. Leading manufacturers of plastic bags give a consistent view of the need for a technical solution for control and monitoring.

Another reason why no satisfactory solution has been found is the fact that the quality of the welding joint in itself is difficult to monitor because the measurable differences between a satisfactory and a deficient welding joint are so small. The welding method used in plastic bag manufacturing is usually a resistive hot-wire type welding. The plastic films are heated and pressed together which closes the bag at the bottom. The welding causes a change of the amount of the material or the properties of the material in the welding joint. Usually this means a thinning of the plastic film, which then represents a measure of the welding joint quality, but there are also welds that are quite satisfactory without any change of the material thickness. If the thinning is too small the welding might be too weak and if it is too big a burn-through might occur.

A typical welding can look like this: Plastic film thickness: 0.2 mm
Plastic film dielectric constant: 3
Welding width type: 1 mm
Welding thinning type: 50%

It has previously been tried to measure the quality of the welding joint by means of optics, ultrasound and other techniques. Such techniques have not been working satisfactory, specifically due to a significant variation of the plastic material. Furthermore, carbon black additives in some of the plastics make the optical methods difficult to use.

None of the previously tried techniques have lived up to the high standards of welding joint control, which requires that a high-resolution and stable measuring signal is obtained during high speed. In summary, it is required that the measuring signal from the sensor has a high resolution, is stable and submitted at high speed. High speed in this context means a speed of 1-3 msec. The sensor should also be wear resistant, it should not disturb the environment and it should not in itself be disturbed by the environment.

In WO 2006/091161 A1 it is described a previous attempt to solve the problem based on microwave technique. In this patent publication it is generally described a system to determine the properties of a material, for example in plastic bag manufacturing. The system comprises means for emitting and measuring microwave radiation and wherein the microwave radiation is used to determine the properties of the material. However, this system has not worked satisfactory, probably due to the high resolution and high sampling rate required to detect the plastic weldings in a measuring gap.

One object of this invention is to provide a plastic welding sensor that is sufficiently sensitive to detect the small material changes that occur in a defective welding joint and which is further arranged to provide this detection at a higher speed compared to similar traditional systems.

Another object of the invention is to provide a sufficiently strong and stable measuring signal that can be interpreted to generate useful information for the user, i.e. in this case provide a warning for defective welding joints.

It is also an object of the invention to reduce/eliminate source of errors of different kinds in the measuring signal, depending on the industrial environment or the like, thus ensuring a higher precision with respect to the result analysis and/or facilitating the analysis work required for the interpretation of the result.

A further object of the invention is to provide a modular sensor made up of substantially standard components that can be mounted in a simple but rugged casing adapted to withstand demanding industrial environments.

A prerequisite for achieving these objectives is a sufficiently high measurement frequency, i.e. that a sufficiently number of measurement values can be generated when a welding is passing. A high impedance in the electrodes further requires a sufficiently high frequency to be driven at the high impedance and a high voltage on the drive electrodes.

In order to reduce noise and interference a narrow bandwidth is required throughout the system. Furthermore, a differential measurement (difference signal between measurement and reference electrode) that only detects differences in the material at high measuring speed.

A further prerequisite for achieving the above objectives is that the detector is phase locked for high dynamics and suppression of interference.

In summary, the following requirements should be fulfilled for a safe and satisfactory function of the sensor:

1. High frequency
2. High voltage on the drive electrodes
3. Narrow bandwidth throughout the system
4. Differential measurement
5. Phase locked detector

According to the invention the material changes in the welding joint is measured by means of a measuring gap comprising two electrodes arranged on opposite sides of the plastic film. The sensor comprises at least two different oriented measuring gaps which measure changes in the dielectric properties of the material as the welding joint passes through the sensor. The measuring gaps are providing a measuring signal proportional to the material change in the welding joint. The electrodes are arranged such that one measuring gap (signal measuring gap) measures the welding profile along the geometry of the welding and the other measuring gap (reference measuring gap) measures an averaged signal over the surface of the plastic film. The electrodes are driven by a stable oscillator with a high measuring frequency in the range of about 100 kHz to 100 MHz.

According to a preferred embodiment of the invention, the sensor is arranged to be mounted at the side of the plastic film along the production line and comprising a first and a second part which parts are arranged to extend above and below the plastic film, respectively, so that two intermediate measuring gaps are formed through which the plastic film is arranged to pass. Both the first and the second part of the sensor is provided with a set of electrodes in the form of a reference electrode and a measuring electrode and wherein the reference electrodes are arranged in the longitudinal direction of the plastic film while the measuring electrodes are arranged perpendicular to the plastic film along the direction of the welding joint.

According to a further preferred embodiment, the electrodes are arranged in a half bridge (half Wheatstone bridge) wherein the electrode gaps have approximately the same average impedance.

According to a further preferred embodiment, an electrostatic shield is arranged between the primary and secondary sides of a coupling transformer in order to minimize undesired capacitive crossover through the system and reduce the effect of especially low frequency conduit interference as otherwise the signal might be lost in noise.

In the following the invention will be described more in detail with reference to the accompanying drawings, wherein

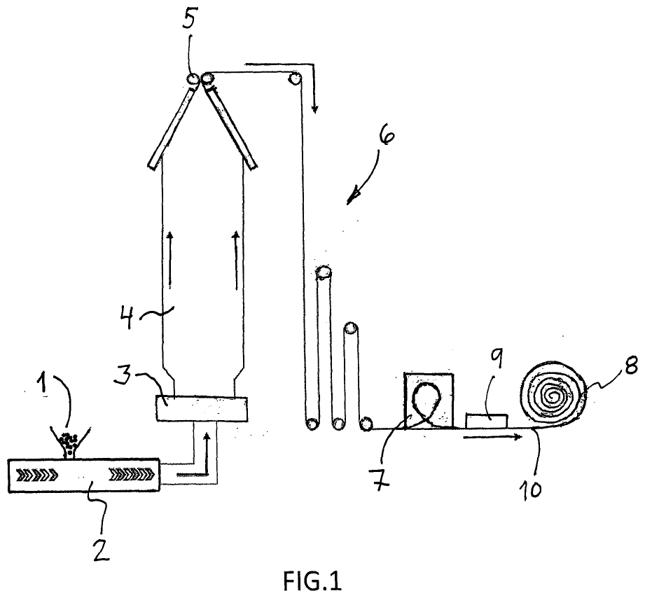

FIG. 1 shows a general overview of the manufacturing of plastic bags,

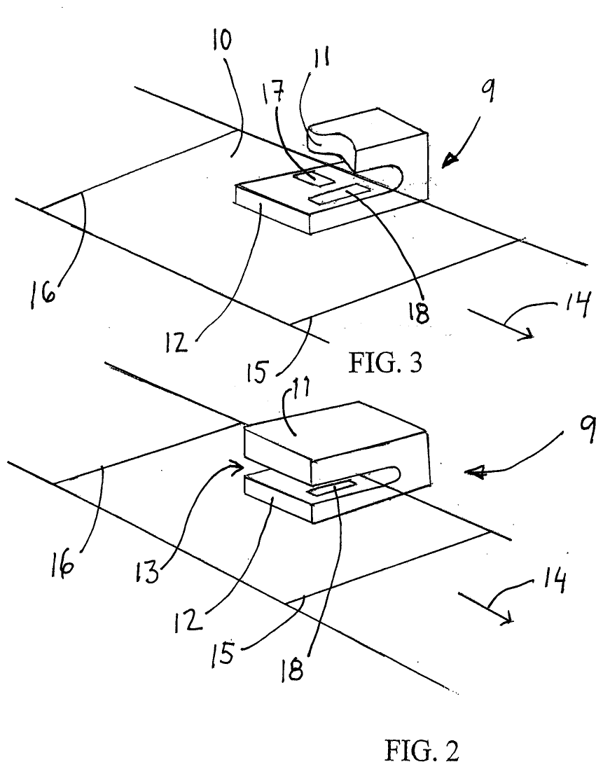

FIG. 2 is a schematic view of a modular sensor adapted to be mounted at the side of the plastic film along a production line,

FIG. 3 is a schematic view of the sensor partially cut to illustrate the location of the electrodes,

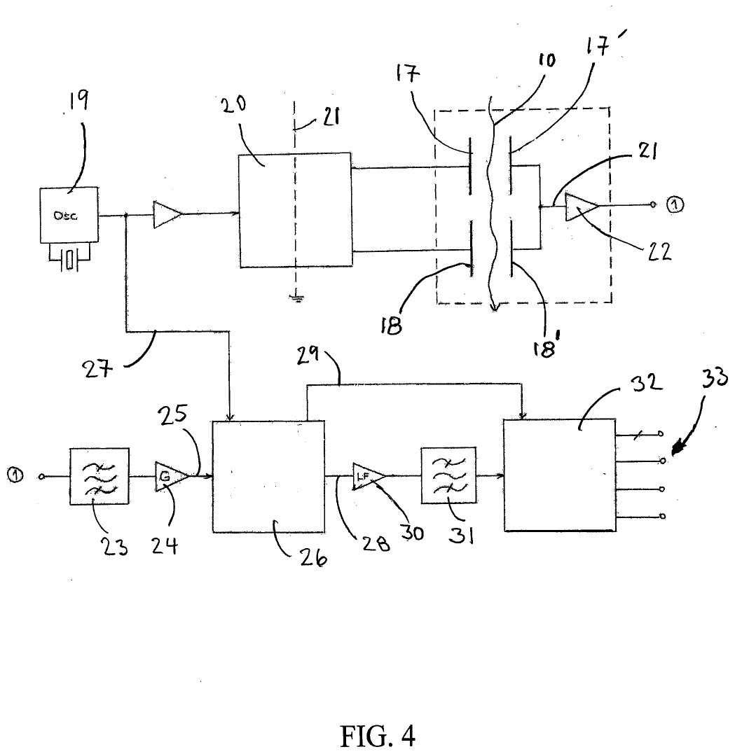

FIG. 4 shows a basic block diagram of a plastic weld sensor according to the invention,

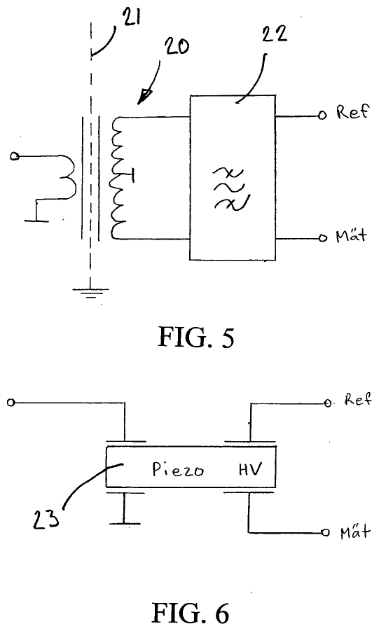

FIG. 5 shows an electrostatic shielded drive transformer,

FIG. 6 shows an alternative embodiment in which a piezoelectric transducer is used for the drive signals,

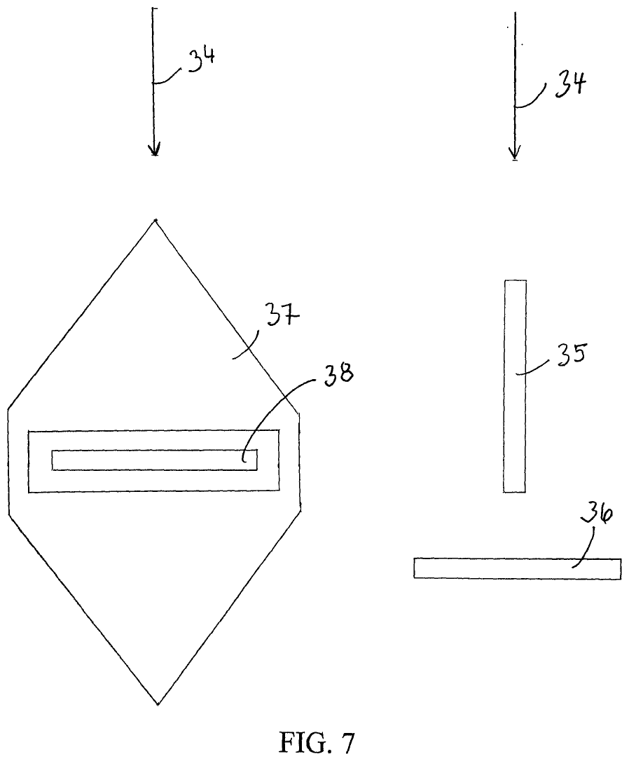

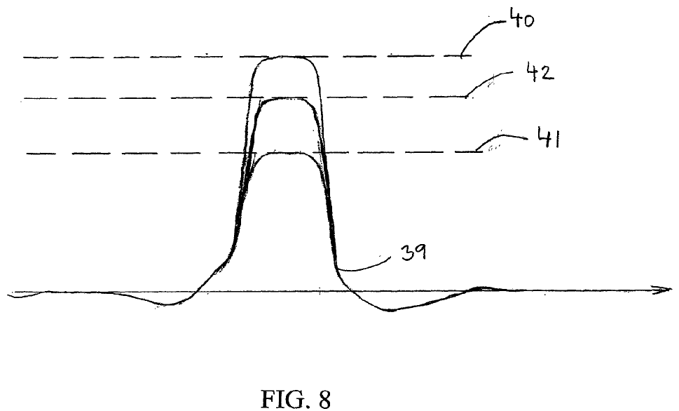

FIG. 7 shows a couple of examples how the electrodes could be located geometrically in the sensor relative to the plastic film, and

FIG. 8 shows an example of evaluation of a signal processed measuring signal from a sensor according to the invention.

FIG. 1 shows a principle view of the manufacturing process of plastic bags on roll according to known technique. In the production of plastic bags in an endless rolling line, so-called rip-off model, film blowing is used as a method for manufacturing plastic films in varying thicknesses. Traditionally, ethylene and propylene plastics are the most common materials, although biodegradable materials are used more and more.

In film blowing the plastic material in the form of granulate is supplied to a heated cylinder (extruder) 2 and melted. A screw rotates and feeds the melt to a cooled nozzle 3 at the end of the cylinder. The plastic melt is then cooled into a semi-state condition at the passage of the nozzle. There the material assumes the shape of a tube/hose 4. After blowing to a balloon shape, the hose is then fed vertically up to two clamping rolls 5 and further on to peel-off and roll-on means 6.

By varying the shape of the gap opening, the amount of melt pressed through the mould, the inflation of the “balloon” to the desired diameter and the rate of peel-off rolling, it is possible to control the thickness of the film as well as the degree of orientation of the molecular chains in the plastic material longitudinally and transverse. By varying the orientation of longitudinal and transverse proportions films of different strengths can be obtained, i.e. films adapted to different applications. Depending on the desired properties of the film, different additives might be used, for instance to reduce the risk for plastic sticking together, providing a smoother surface, or protecting the plastic from ultraviolet radiation.

The manufacturing of the plastic bags is finished by feeding the flattened plastic hose into a so-called conversion machine 7, in which the plastic film is welded, so that the bag is provided with a bottom, and perforated to the desired length for easy tearing away from a roll. This technique is used for all bags, ranging from big bags, bin bags, garbage bags, knot bags or the like, until small bags for household use, such as freezer bags. In a final step the plastic bags are rolled up on a roll 8 and packaged.

The manufacturing process of plastic bags takes place at high speed. The plastic web can typically have a speed of 1-3 msec in this type of manufacturing process.

As mentioned in the introductory portion of the specification there is a need for checking the quality of the products in order to reduce the disposal of finished plastic bags. Primarily, it is the quality of the welding joints that causes the problems with the high disposal of plastic material. The problem has been known for a long time without any satisfactory solution so far.

According to the invention a sensor has been developed which sensor is able to monitor in real time the quality of welding joints at high speed production of plastic bags on a roll. High speed means here speeds of several msec. The sensor is arranged to be mounted at the side of the plastic film along the production line for measuring material changes in the welding joint. The sensor comprises at least two electrodes for measuring changes in the dielectric properties of the material as the welding joint is passing through the measuring gap of the electrodes and provides a measuring signal which is proportional to the material change in the welding joint. The sensor 9 is preferably modular and adapted to be mounted at the side of the plastic film 10 along a production line after the conversion machine 7 as schematically indicated in FIG. 1.

The sensor 9 is arranged in a rugged shell and made in the form of a measuring fork with a first and a second part 11 and 12, respectively, arranged to extend above and underneath the plastic web, respectively, so that an intermediate measuring gap 13 is formed and through which the plastic film 10 can freely pass for a contact-less measurement, a “fork-like” appearance, see FIG. 2. In the figure, the direction of the plastic film 14 is shown and two welding joints 15,16 to be monitored by the sensor.

Both the first and the second part of the sensor is provided with an electrode setup in the form of a reference electrode 17 and a measuring electrode 18. The reference electrode is arranged in the longitudinal direction of the plastic web, while the measuring electrode is arranged perpendicular to the direction of the plastic web, in the direction of a welding joint, see FIG. 3 showing the sensor partially cut away. The function of the electrodes is described more in detail below in connection with FIG. 4.

FIG. 4 shows a basic block diagram of a plastic weld sensor according to the invention. The plastic weld sensor has a reference measuring gap between reference electrodes 17 and 17′ and a signal measuring gap between measuring electrodes 18 and 18′. The plastic film 10 passes through these two measuring gaps as illustrated schematically in the figure. The electrodes are arranged in an electrical half bridge in such a way that one of the measuring gaps (the signal measuring gap) is measuring the weld profile across the plastic film web and the other measuring gap (the reference measuring gap) is measuring averaging longitudinally along the plastic web. How the electrodes are arranged geometrically is illustrated more in detail in FIG. 7 below.

The electrodes are driven by a stable oscillator 19 having a high stable frequency in order to obtain a sufficient sensitivity when the measuring impedances are high. At high frequencies it is an advantage to measure the dielectric properties instead of using capacitive and impedance sensors. A high measuring speed is obtained and also the noise is low which is a prerequisite for a sufficiently strong and stable measuring signal. Typically, the measuring frequency is within the range of 100 kHz to 100 MHz depending on the application.

The oscillator 19 is connected to a shielded drive transformer 20 arranged to provide two output signals, one to the reference electrode 17 and one to the measuring electrode 18 in the first electrode setup, The output signals are 180 degrees phase shifted relative to each other. The drive transformer comprises a tuned centre grounded transformer coupling with primary and secondary sides and arranged to create a high drive voltage and impedance matching of the high impedance measuring electrodes. If for instance there is a difference between the reference electrode 17 and the measuring electrode 18 so that the impedance difference causes an unbalance in the bridge coupling, it can be balanced with a slight change in the balancing of the drive transformer.

Preferably, the drive transformer 20 is provided with an electrostatic shield, as illustrated in FIG. 5. The electrostatic shield 21 is arranged between the primary and secondary side of the transformer coupling in order to minimize undesired capacitive crossover through the system and reduce the effect of particularly low-frequency conducted interference, otherwise the signal might be lost in noise. The output signals (Ref, Mat) to the electrodes are filtered by a narrow band-pass filter 22 in order to reduce noise (outside the band-width of the signals).

FIG. 6 shows an alternative embodiment in which a piezoelectric converter 23 is used to deliver the two output signals (Ref, Mat) to the reference electrode and the measuring electrode, respectively. Electrostatic as well as piezoelectric components of the type used here are previously known per se for those skilled in the art and are therefore not described in any detail here.

The other electrode setup illustrated in FIG. 4, on the other side of the plastic film 10, comprises in the same way a reference electrode 17′ and a measuring electrode 18′. The reference electrode 17′ is arranged to provide an average over a large area, while the measuring electrode 18′ has a geometry to make it most sensitivity for the design of the welding joint in the desired measuring direction. The summarized output signal 21 from the electrodes 17′ and 18′ has an average value close to zero over time to allow for a most possible amplification without any risk for signal bottom in a subsequent step, which otherwise happens when the signal has a phase difference of 180 degrees. Thus, there is substantially no output signal from a homogeneous film.

The summarized output signal 21 is emitted to an amplifier 22, for example a high impedance FET amplifier with an input impedance much greater than the impedance of the measuring gaps. Such an amplifier usually has a very low amplification (impedance converter) which means that a subsequent band-pass filter 23 as well as an additional amplifier 24 are required before the measuring signal 25 is forwarded to a detector 26.

The detector 26 is arranged to remove unwanted noise outside the band-width of the measuring signal which is necessary for a desired signal to noise ratio. Thus, it is important that the detector is a narrow-band type detector. Various types of detectors are previously known for those skilled in the art, such as phase-locked detectors, mix detectors, synchronous detectors etc, and are not described in any detail here. A common feature for this type of detectors is that they usually provide a narrowband solution, which is necessary in our case. Preferably, a phase locked detector is used for high dynamics and suppression of interference.

Input signal to the detector 26 in FIG. 4, in addition to the measuring signal 25, is a signal 27 from the oscillator 19. Output signal from the detector 26, in addition to the low frequency measuring signal 28, is a signal 29 indicating the phase position. The measuring signal is emitted to a low-frequency amplifier 30 and a band-pass filter 31 for the desired frequency range.

The sensor further comprises signal processing means 32 for evaluating the measuring signal for detecting defective welding joints. For example, in case of poor quality an alarm can be triggered to alert the operator. This means that the investigating work is minimized and the operator can directly take steps to restore the welding quality, for instance by an adjustment of the welding station. Furthermore, the sensor might be equipped with an appropriate software for communicating with the plastic bag manufacturing control system.

It could also be said that one object of the signal processing means 32 is to verify the welding quality and other material changes, such as perforations and other defects, in the plastic film. Furthermore, there is information about the position of the plastic film track transverse to the film direction which could be used to count the number of products or indicate interruptions in the plastic web. The output signal from the signal processing means indicates the desired variable and could be used for a direct alarm or, alternatively, be communicated to information buses or telephone systems in the manufacturing facilities. The signal might also be used for controlling previous process steps. These different functions of the output signal are indicated in FIG. 4 by means of four signal outputs 33.

In FIG. 7 it is illustrated by way of some examples how the electrodes could be positioned geometrically in the sensor relative to the plastic web. The moving direction of the plastic web is indicated by the arrows 34. In the first embodiment the rectangular, elongated reference electrode 35 is positioned along the longitudinal direction of the plastic web thereby providing an average value, while the rectangular measuring electrode 36 is positioned transverse to the web so as to be more sensitive to a passing welding joint. The dimensions of the measuring electrode might be relatively small. In a typical case the electrode width is 3 mm and the electrode length 20 mm.

In an alternative embodiment the reference electrode 37 is more wide, wider for covering a greater portion of the width of the plastic film, with an elongated wedge shape to surround the measuring electrode 38 which also in this case has a rectangular and elongated geometry and positioned transverse to the plastic web.

FIG. 8 shows an example of evaluation of a signal processed measuring signal 39 from a sensor according to the invention. The signal curve gives an indication of the quality of the welding joint by measuring material changes that arise during the welding process. The material changes could either be in the form of a change of the geometrical shape of the welding, or a change of the dielectric properties of the material. In the figure it is illustrated how the amplitude of the measuring signal is changed at a welding joint passage. For example, the sensor might have two detection levels or, as an alternative, an approved operating range outside which the sensor should alarm. In the figure it has been indicated limit values for a too strong welding effect 40, so that in a typical case a burn-through of the plastic film might occur, and limit values 41 for an insufficient welding effect which means a poor welding joint. Between these two limit values a good, acceptable welding joint 42 is indicated.

The invention is not limited to the examples described above but may be varied within the scope of the accompanying claims. For example, it is realized that the location of the sensor along the production line can be varied, but it should be located after the conversion/welding machine on a section where the bending force and velocity of the plastic film is as constant as possible. Furthermore, it is realized that the geometric design of sensor housing and electrodes could be varied.

It is also apparent to those skilled in the art that the sensor housing could be divided into two independent parts with a cable connection and alternative mechanical couplings in a plastic machine. One example is the case of measuring in the middle of a plastic web in cases where fork measuring is less favourable and a stable external mechanism of for instance steel profiles might be better. Also. in some cases two or more detectors might be required to get the desired benefit. Furthermore, the sensor could be rotated 90 degrees to be used for assessing longitudinal welding joints.

1. Sensor for real time monitoring the quality of welding joints in a plastic film at the production of plastic bags on roll by measuring material changes in the welding joint, whereby the sensor comprises at least two electrode pairs forming measuring gap arranged to measure changes in the dielectric properties of the material when the welding joint is passing through the measuring gap of the electrodes and provide a measuring signal proportional to the material change in the welding joint, and that the sensor further comprises signal processing means for evaluating the measuring signal in order to detect defective welding joints in the plastic film, characterized in that the electrodes are arranged in such a way that a first measuring gap, the signal measuring gap, is arranged for measuring the welding profile, and at least a second measuring, the reference measuring gap, is arranged to provide an average of the plastic film measuring signal and whereby the electrodes are driven by a stable oscillator with a high measurement frequency in the order of 100 kHz to 100 MHz and that the sensor is arranged at the side of the plastic film along the production line and is provided with a first and a second part which parts are arranged to extend above and beneath the plastic film, respectively, so that an intermediate measuring gap is formed and through which the plastic film is arranged to pass, and wherein the first as well as the second part of the sensor is provided with a set of electrodes forming said reference measuring gap and a signal measuring gap and wherein the reference electrodes are arranged along the longitudinal direction of the plastic film and the measuring electrodes arranged perpendicular to the plastic film.

2. Sensor according to claim 1, characterized in that the electrodes are arranged in a half electric bridge (half Wheatstone bridge) where the electrode gaps have approximately the same average impedance.

3. Sensor according to claim 1, characterized in that the oscillator is connected to a shielded drive transformer arranged to provide two output signals, one signal to the reference electrode and one signal to the measuring electrode to create a high drive voltage and impedance matching of the electrodes.

4. Sensor according to claim 3, characterized in that the output signals are 180 degrees phase shifted relative to each other.

5. Sensor according to claim 3, characterized in that an electrostatic shield is arranged between the primary and secondary sides of the drive transformer in order to minimize unwanted capacitive crossover through the system and reduce the effect of primarily low frequency conducted interference to prevent that the signal is hidden in noise.

6. Sensor according to claim 1, characterized in that the measuring signal is detected by means of a narrow band detector, for instance a phase locked detector, for high dynamics and suppression of interference.

7. Sensor according to claim 1, characterized in that it is arranged to be mounted in pairs, one on each side of the plastic film, along a production line for plastic bag manufacturing.

8. Sensor according to claim 1, characterized in that the reference electrodes are arranged to provide an average over a big area, while the measuring electrodes have a geometry to provide a most possible sensitivity for welding profile in the desired measuring direction.

9. Sensor according to claim 1, characterized in that the signal processing means are arranged to indicate a desired variable and provide an alarm signal or, as an alternative, connect to information buses or telephone system in the production facility or used to control a previous process step, etc.

10. Sensor according to claim 1, characterized in that the signal processed measuring signal provides a measure of the quality of the welding joint by measuring the material changes that arise during the welding process, and whereupon the material changes could either be in the form of a geometrical change of the welding shape or a change of the dielectric properties of the material.

11. Sensor according to claim 10, characterized in that the signal processing means has two detection levels for indicating a too strong welding effect and an insufficient welding effect, respectively, and between which two detection levels an approved operating range is indicated and outside which range the sensor is arranged to make an alarm.
